(19) United States
(12) Patent Application Publication
(10) Pub. No.: US 2012/0328543 A1
(43) Pub. Date: Dec. 27, 2012

(54) USE OF A CATIONIC, ADVANTAGEOUSLY AMPHOTERIC, SURFACTANT FOR THE PREPARATION OF AN ANTIFUNGAL SOLUTION THAT CAN BE APPLIED TO THE NAIL

(75) Inventors: Claire Mallard, Mougins (FR); Nathalie Willcox, Magagnosc (FR)

(73) Assignee: GALDERMA PHARMA S.A., Lausanne 30 Grey (CH)

(21) Appl. No.: 13/516,892

(22) PCT Filed: Dec. 17, 2010
(86) PCT No.: PCT/EP10/70083

§ 371 Date: Sep. 4, 2012

(30) Foreign Application Priority Data
Dec. 18, 2009 (FR) 0959185

(51) Int. Cl. A61K 31/137; A61P 31/10

(52) U.S. Cl. 424/61

(57) ABSTRACT

The use of a cationic surfactant, or an amphoteric surfactant, for the preparation of a composition comprising an antifungal agent in the form of an acid salt, intended to be applied to the nail is described.

### TECHNICAL FIELD

The present invention relates to a composition that improves the penetration through the nail of an antifungal agent that is in the form of an acid salt, such as for example terbinafine hydrochloride. More precisely, it is proposed to formulate the antifungal agent in the presence of a cationic, advantageously amphoteric, surfactant such as coco betaine.

Such pharmaceutical or dermatological compositions are particularly useful for the treatment, in humans and animals, of onychomycoses in particular due to dermatophytes or to Candida.

### PRIOR ART

The nails are frequently the site of onychomycoses especially dermatophytic or candidal onychomycoses.

Although the treatment of these pathologies using antifungal agents is carried out in a favoured manner transungually, the very rigid structure of the nail makes treatment difficult, hence the interest in optimizing the composition of the product so as to be able to impregnate the nail as deeply as possible.

During the formation of the nail plate, the basal cells of the matrix grow, the nuclei of the cells break up, the cytoplasms fuse in order to form highly individualized larger and keratinized cells that constitute the granular layer.

The nail plate is constituted of dead, keratinized and adherent cells, with no nucleus, but with thick membranes. It essentially contains α-keratin and is made up of three layers: the dorsal and intermediate layers, resulting from the matrix, and the ventral layer, resulting from the nail bed (FIG. 1).

The dorsal part is formed from a few layers of cells rich in hard keratin constituting the granular layer. The intermediate part is constituted of cells rich in keratin that is softer with disappearance of the granular layer, and constitutes three quarters of the total thickness of the nail. The ventral part is formed from one or two layers of cells rich in soft and hyponychial keratin; there is still no granular layer.

The nail is essentially composed of keratin, a scleroprotein rich in sulphur-containing amino acids. From the morphological point of view, keratin fibres are for the most part oriented perpendicular to the growth of the nail, in a plane parallel to the surface of the nail.

The keratin chains are linked together by bonds of various types: hydrogen bond, peptide bond, polar bond and disulphide bond (FIG. 2). These bonds may be attacked by various stresses: chemical agents, alkaline agents, oxidizing agents, thioglycolates for the disulphide bridges; by disintegration by strong acids or bases for acid-base union; or rupture of the hydrogen bridges by water molecules.

The chemical composition of the nail is closer to that of the hair than that of the skin. The fatty lipophilic compounds of the nail represent only 0.1 to 1% of the constituents of the nail. This is essentially cholesterol, which has a plasticizing role.

Water is present between 15 and 18% and may even reach 25%. The water content of the nail is above all dependent on the degree of hygrometry. At saturation, it may reach one third of the dry weight of the nail. It is thus easy to understand that the hydrophilic molecules penetrate the nail plate more easily than lipophilic molecules.

There are also trace elements in the nail: zinc, iron, manganese, copper, etc.

Sulphur makes up 5% of the weight of the plate, which is particularly rich in sulphur-containing amino acids, mainly cystine and arginine.

The treatment for onychomycosis comprises various approaches:
surgery, which consists in debriding the lesion down to the healthy nail;
topical antifungal treatments; and
systemic antifungal treatments.

Most oral treatments that have a systemic action are long and are not without significant side effects.

The option of topical treatment proves much less toxic, but in order to be effective, requires the antifungal agent to be capable of penetrating through the hard keratin of the nail and reach the nail bed at a sufficient concentration in order to be able to destroy and eradicate the pathogen, Trichophyton rubrum.

The formulation of the antifungal molecules currently available however poses a certain number of problems:
generally, their solubility is very low in water, and therefore makes the formulation difficult at effective concentrations of active principle;
the composition of the excipient is not systematically adapted to the chemical composition of the nail in order to be able to reach the nail bed; and
the penetration through the nail in order to reach the nail bed is often difficult.

Thus, the topical treatments that exist for onychomycosis are in the form of film-forming varnishes which are not very effective as regards penetration of the active principle through the keratin of the nail.

Furthermore, the penetration of an active principle through the human nail is a phenomenon that is not widely described in the literature. Although there are numerous molecules that are known to promote penetration through the skin, these molecules are not very suitable for an ungual application.

By way of example, the antifungal agent terbinafine is available in the form of terbinafine hydrochloride (terbinafine HCl) of the following formula (A):

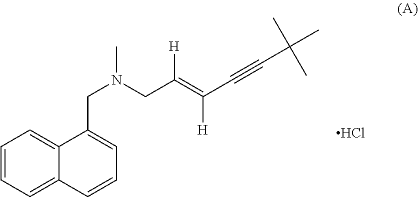

Due to its cationic nature, this molecule, besides its very low solubility in water (5.32 μg/ml; Narendra Kumar & al. Asian J Pharmaceutics, 2(3), 154-158, (2008)), has a great affinity for keratin. Terbinafine HCl therefore binds very easily to keratin from the upper layers of the nail and due to this fact, only a small proportion actually penetrates down to the nail bed.

With regard to the prior art, the (“Drug delivery to the nail following topical application”, S Murdan, Int J Pharm 236 (2002) 1-26), numerous solutions have been investigated in order to improve the problem of penetration through the nail:
nature of the excipient and in particular the presence of water;
importance of organic solvent such as DMSO;
influence of pH and the ionic charge of the excipient on the ionization of the antifungal active principle;
influence of absorption promoters with various modes of action on the nail matrix.

Therefore, there is a need to find novel formulations for antifungal agents that allow better penetration through the nail by an effective amount of active principle.

### SUMMARY OF THE INVENTION

The present invention is based on the demonstration by the Applicant that the presence of a cationic, advantageously amphoteric, surfactant makes it possible to increase the penetration of an antifungal agent that is in the form of an acid salt, such as terbinafine HCl.

In other words, the invention consists in reducing the binding sites of the antifungal agent to the keratin using a molecule that acts as a competitor for the antifungal agent with respect to keratin.

Thus, it has been shown within the context of the present application that the presence of such a surfactant:
in no way affected the solubilization at high concentration (for example 10%) of the antifungal agent in an aqueous solution;
gave the composition properties of compatibility with the biological composition of the nail; and
made it possible to increase the penetration and the diffusion of the active agent through the nail.

The expression “penetration of the active agent” is understood to mean the ability of this agent to go into the nail matrix.

The expression “diffusion of the active agent” is understood to mean the ability of this agent to reach, starting from the matrix, the nail bed under the matrix.

Without wishing to be tied to any one theory, it is assumed that the surfactant, due to its amphoteric nature and in particular its cationic form, binds to the keratin thus enabling Terbinafine HCl, at the acid pH of the composition (pH below the pKa of Terbinafine, 7.10 (Novartis Pharmaceuticals Canada Inc., Prescribing Information, ^{Pr}Lamisil (terbinafine hydrochloride), 2008, page 18)) and advantageously at a pH between 3 and 6, and preferably between 3 and 5, to diffuse through the nail matrix and to reach its site of action, the nail bed.

Thus, and according to a first aspect, the present invention describes, for the first time to the knowledge of the Applicant, the use of a cationic, advantageously amphoteric, surfactant for the preparation of a composition in the form of a solution comprising an antifungal agent in the form of an acid salt, intended to be applied to the nail.

According to another aspect, the present invention targets a pharmaceutical composition intended to be applied to the nail comprising:
an antifungal agent that is in the form of an acid salt, advantageously a hydrochloride;
a solvent system; and
a cationic, advantageously amphoteric, surfactant,
   said composition being in the form of a solution.

The first component of this composition is therefore an antifungal agent that is in the form of an acid salt, advantageously a hydrochloride.

Generally, any antifungal agent present in cationic form within the composition can be used under the conditions of the invention.

The antifungal agents of interest that are more particularly targeted by the present invention are those from the class of allylamines or morpholines, allylamines being preferred. Indeed, antifungal agents from the class of allylamines, in particular terbinafine or naftifine, and also those from the class of morpholines, in particular amorolfine, are promising compounds in the antifungal fight. Their presumed or demonstrated mode of action would take place through inhibition of ergosterol, a specific constituent of the wall of fungal cells, in particular via inhibition of squalene epoxidase.

Within the class of allylamines, mention may be made, in particular, of terbinafine hydrochloride and naftifine hydrochloride, the respective formulae of which, (A) and (B), are the following:

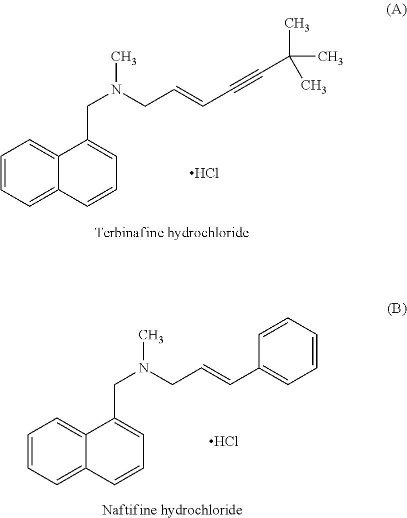

Among the molecules of this class, terbinafine is preferred.

Alternatively, the antifungal agent may belong to the class of morpholines, in particular amorolfine, for which similar problems are faced.

In practice, the antifungal agent such as has been defined above represents more than 5%, preferably at least 8%, or even at least 10% (w/w) of the total composition. It is thus possible to envisage up to 15%, or even 20%, of this agent in the composition. Obviously, it is possible to envisage a mixture of antifungal agents, optionally of different classes in order to increase the efficacy.

As already stated, these molecules have the drawback of being almost insoluble in water and of diffusing very little in the nail, probably due to their interaction with keratin.

The second component of the composition according to the invention is the solvent system which is advantageously a ternary system. Advantageously, it is constituted of the following ingredients:
water;
at least one C2-C8 alkanol with a straight or branched chain, advantageously ethanol; and
at least one glycol (having free hydroxyl functional groups), advantageously propylene glycol.

More advantageously still, the amount of total water represents more than 30% by weight of the composition, advantageously more than 33%, even more than 35% or even more than 40%. This large amount of water in the formula gives the product a considerable hydrophilic nature. Indeed, since the nail is a hygroscopic hydrophilic matrix, it swells in the presence of water which facilitates the diffusion of the active principles.

The expression “total water” is understood to mean the amount of water introduced as is into the composition, added to the amount of water originating from the various solvents and/or excipients of the composition when they contain some thereof.

This high water content is extremely advantageous due to the targeted transungual application. Moreover, it has been shown within the context of the invention that despite the low solubility in water of the antifungal molecules in question, the solvent system proposed allows a solubilization at high concentration of the molecules of interest, including in the presence of the cationic or amphoteric surfactant according to the invention.

Besides the high water content, the ternary solvent system advantageously contains a short-chain alcohol, and more precisely at least one C2-C8 alkanol having a straight or branched chain, preferably ethanol, isopropanol and n-butanol. Ethanol is particularly preferred. A mixture of various alcohols may also be envisaged.

Finally, this ternary solvent system comprises at least one glycol. The term “glycol” is understood here to mean a compound that has at least two hydroxyl functional groups. Glycols for which the two hydroxyl functional groups are free, that is to say that they are not involved in an ether or ester bond, are more precisely targeted by the invention. Mention may be made, for example, of propylene glycol, butylene glycol, pentylene glycol, hexylene glycol, ethylene glycol and polyethylene glycols. Propylene glycol is preferred. A mixture of various glycols may also be envisaged.

Advantageously, the ternary solvent system represents at least 60%, or 70%, 80% or even 90% (w/w) of the total composition.

In addition, and advantageously, the proportion of alcohol is greater than or equal to that of glycol. More advantageously still, the proportion of total water is greater than that of glycol.

The third component of the composition according to the invention is therefore a surfactant of cationic, or even amphoteric, nature capable of entering into competition with the antifungal agent in the form of an acid salt for binding to keratin. The latter is present in the structure of the various layers of the nail in the form of fibres and is negatively charged.

According to the invention, the cationic or amphoteric surfactant is therefore introduced for the effects it produces in situ on the bioavailability of the active principle in the nail, especially on the bioavailability of terbinafine HCl, through the keratinized ungual nail plate, skin appendages and skin.

By definition, a cationic surfactant is a surfactant in which the hydrophilic part is positively charged. It releases a positive charge (cation) in aqueous solution. It has bacteriostatic and emulsifying properties and exhibits an affinity with the negatively charged keratin with which it will combine.

An amphoteric surfactant is a surfactant in which the hydrophilic part comprises a positive charge and a negative charge, the overall charge being zero at the isoelectric point. Depending on the pH of the medium which it is in, it releases a positive ion and a negative ion. At alkaline pH, it behaves as an anionic surfactant, and at acid pH, it behaves as a cationic surfactant.

Within the context of the invention, in so far as the antifungal agent is in the form of an acid salt, the composition is therefore at acid pH below the pKa of the antifungal agent, preferably at a pH between 3 and 6, preferentially between 3 and 5, and the positively charged amphoteric surfactant then acts as a cationic surfactant.

As the cationic surfactants that can be used according to the invention, mention will be made, non-limitingly, of:

quaternary ammoniums,

in which the counterion may be
chloride, bromide, phosphate, hydroxide, methosulphate, sulphate or a carboxylic acid anion;
   in which the substituents of the nitrogen may be
saturated or unsaturated, optionally hydroxylated, alkyl chains having 1 to 20 carbons, the hydroxyl functional group possibly being esterified, it being possible for these chains to optionally be substituted, to originate from defined compounds, or else to be mixtures resulting from natural products;
optionally substituted aromatic groups, rings, in particular aromatic rings, for example pyridine, which are optionally substituted;
mixtures of these various categories;
themselves substituted by a quaternized or non-quaternized amine functional group;

amines, which may be protonated depending on the pH and amine salts in which the nitrogen bears the substituents mentioned previously and/or hydrogen, these products being used under conditions where they are cationic;

derivatives of betaine or of amino acids, under pH conditions that make them cationic, optionally substituted by the groups mentioned previously.

Advantageously, the amphoteric surfactants used have a structure of a betaine derivative corresponding to the general formula (I) below:

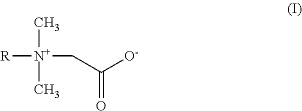

in which R represents an alkyl radical or an R′CO—NH(CH₂)₃— radical, R′ representing an alkyl radical.
The expression “alkyl radical” is understood to mean a saturated, linear or branched hydrocarbon-based chain. Among the alkyl radicals, those comprising from 1 to 20 carbon atoms will be preferred.

Among the betaine derivatives, preference will more particularly be given to the amphoteric surfactant known under the trade name Dehyton® AB30 or else Lauryl Dimethylaminoacetic Acid Betaine corresponding to the general formula (I) in which R represents the lauryl radical. This molecule is commonly known as coco betaine.

This molecule is, for example, sold by Cognis as a 30% aqueous solution under the name Dehyton® AB30.

As additional examples, mention may be made of other betaines such as cetyl betaine or else cocamidopropyl betaine.

The cationic or amphoteric surfactant represents at least 0.1% by weight of the composition. It may represent up to 10%, or even 15% by weight of the composition without disturbing the solubilization of the antifungal agent at high concentration.

According to one particular embodiment, the cationic and/or amphoteric surfactant is the sole surfactant in the composition according to the invention. This excludes the simultaneous presence of non-ionic surfactants and also of anionic surfactants.

Advantageously, the composition according to the invention also comprises a texturing agent from the class of celluloses, for example alkyl cellulose derivatives, in particular methyl celluloses, ethyl celluloses, propyl celluloses and hydroxyalkyl celluloses, such as those sold under the name KLUCEL, advantageously hydroxyethyl cellulose (Natrosol HHX250) or hydroxypropyl cellulose. This texturing agent that adjusts the viscosity of the composition allows a comfortable application thereof, especially to the nail. Moreover, the control of the viscosity makes it possible to avoid the too rapid evaporation of the solvents and thus makes it possible to control the recrystallization of the active principle.

Furthermore, the composition according to the invention may also contain at least one additive chosen from the group formed by:
preservatives, such as phenyl ethyl alcohol, benzyl alcohol, phenoxyethanol, parabens and derivatives thereof;
antioxidants, such as butylhydroxyanisole (BHA), butylhydroxytoluene (BHT), palmityl ascorbate, α-tocopherol and/or its esters;
dyes, fillers or pigments, such as the titanium micas commonly used in the cosmetics field for producing nail varnishes;
chelating agents such as disodium EDETATE (EDTA);
emollients such as cyclomethicone; and
other active principles such as an antiseptic, especially acetic acid.

The amounts of each of these additives are easily determined by a person skilled in the art.

Generally, the composition according to the invention may be a gel, a spray, a solution or a foam.

The composition according to the invention is not a varnish. Therefore, it does not contain any film forming agent.

Advantageously, the composition according to the invention is an aqueous composition of solution type. The term “solution” is understood to mean a clear and homogeneous liquid preparation containing one or more substances dissolved in a solvent or mixture of solvents that are miscible with one another. The expression “liquid preparation” is understood to mean a product which flows at room temperature and that has a Newtonian nature or a pseudoplastic flow.

Typically, and by way of illustration, a composition according to the invention comprises:
1% to 20% by weight of terbinafine HCl in solubilized form;
0% to 10% by weight of a texturing agent;
0.1% to 20% by weight of an amphoteric surfactant;
20% to 80% by weight of solvent phase predominantly containing water;
0% to 1% of a chelating agent;
0% to 2% of an antioxidant; and
0% to 20% of additives.

Preferably, the composition is constituted of:
1% to 15% by weight of terbinafine HCl in solubilized form;
0% to 5% by weight of a texturing agent;
0.1% to 15% by weight of an amphoteric surfactant;
20% to 60% by weight of solvent phase predominantly containing water;
0% to 0.5% of a chelating agent;
0% to 1% of an antioxidant; and
0% to 10% of additives.

More preferably still, the composition is constituted of:
1% to 10% by weight of terbinafine HCl in solubilized form;
0% to 2% by weight of a texturing agent;
0.1% to 10% by weight of an amphoteric surfactant;
20% to 55% by weight of solvent phase predominantly containing water;
0% to 0.05% of a chelating agent;
0% to 0.5% of an antioxidant; and
0% to 5% of additives.

Preferably, the composition is constituted of:
1% to 10% by weight of terbinafine HCl in solubilized form;
0.1% to 2% by weight of a texturing agent;
0.1% to 10% by weight of an amphoteric surfactant;
20% to 55% by weight of solvent phase predominantly containing water;
0% to 0.05% of a chelating agent;
0% to 0.5% of an antioxidant; and
0% to 5% of additives.

Thus, the present invention relates to a pharmaceutical or dermatological composition intended for the treatment of onychomycoses.

As already stated, a composition according to the invention is particularly suitable for the treatment of onychomycoses transungually. Thus, it is intended to be applied to the surface of the nail.

### EXEMPLARY EMBODIMENTS

The invention and its attendant advantages will emerge more clearly from the following exemplary embodiments, in support of the appended figures. These are not however in any case limiting.

The following examples relate to formulations in accordance with the invention, of solution type, which make it possible to solubilize at least 5% w/w of terbinafine HCl, while ensuring a comfortable application thereof for the patient.

### KEY TO THE FIGURES

FIG. 1 represents a cross-sectional diagram of the structure of a nail.

FIG. 2 is a schematic representation of the various bonds that exist in the keratin chains in the nail.

FIG. 3 represents the variation in the content of terbinafine hydrochloride (% free terbinafine), as a function of the time, in formula 2 (1.8% coco betaine) relative to the control formula 1 (0% coco betain).

FIG. 4 represents the percentage (%) of solids, as a function of the time, of compositions containing variable amounts of texturing agent (hydroxyethyl cellulose).

FIG. 5 illustrates the evaporation rate of compositions containing variable amounts of texturing agent (hydroxyethyl cellulose).

FIG. 6 illustrates the accumulated amount of Terbinafine HCl that has diffused through nails.

FIG. 7 illustrates the amount of Terbinafine HCl in the nail after 5 days of diffusion.

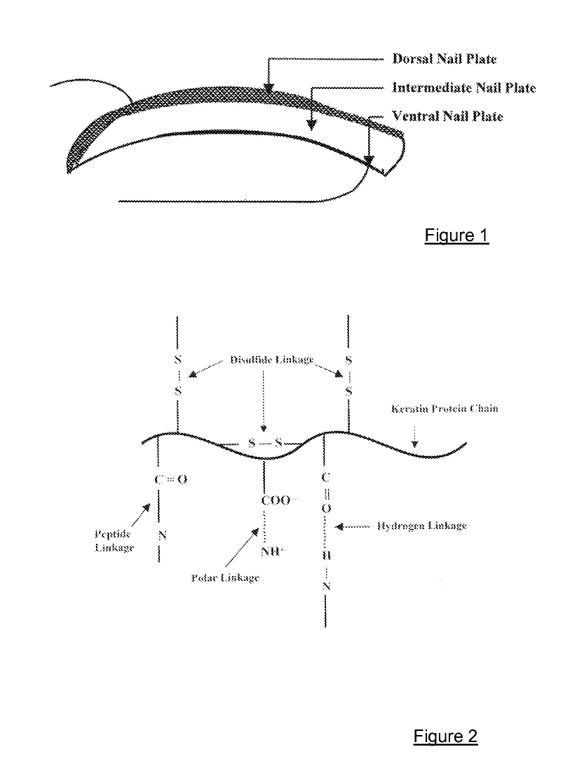

### EXAMPLE 1

### Manufacturing Process and Stability Study

1/Manufacturing Process:

This manufacturing process is carried out simply in a manufacturing beaker by swelling the texturing agent in water. Next the active phase (see preparation below) containing the terbinafine hydrochloride in solution is added. Then, under moderate stirring, the cationic or amphoteric surfactant is added.

a—Preparation of the Aqueous Phase:

Water and the texturing agent are introduced into a beaker and left under stirring in order to obtain a clear, smooth and homogeneous mixture.

b—Preparation of the Active Phase:

In an additional beaker, the active principle is solubilized in the organic, glycol and alcoholic solvents.

c. Final Mixture:

The active phase (b) is incorporated into the aqueous phase (a) and homogenized, then the cationic or amphoteric surfactant is added and the homogenization is continued.

### Viscosity Control Method:

Brookfield LVDVII+viscometer
SC-18+small volume spindle

### Rate: 12 rpm

Time: 1 minute

### Temperature: 25° C.

2/Measurement of the Stability of the Composition:

a—Physical Stability:

The physical stability of the formulations is measured by a macroscopic observation of the formulation at room temperature (RT), at 4° C. and at 40° C., after 1 month, 2 months and 3 months in order to guarantee the physical integrity of the products and to verify the absence of recrystallization of the solubilized terbinafine HCl.

Microscopic analysis is carried out at 4° C. and at room temperature (RT) in order to verify the absence of recrystallization of the solubilized terbinafine HCl.

b—Chemical Stability:

The chemical stability is measured by assaying the active principle using HPLC and the results are expressed as % of the initial content.

### EXAMPLE 2

### Solution Containing 5% of Terbinafine HCl

| | | | Content |
|---|---|---|---|
| | | | (% taking into |
| | | | account the water |
| | | Content | contained in each |
| Ingredients | INCI name | (% w/w) | ingredient) |
| Purified water | Aqua | 39.05 | / |
| Total water | Aqua | / | 39.05 + |
| (purified water + | | | (6.0 × 0.7) = |
| water contained in | | | 43.25 |
| the ingredients) | | | |
| Titriplex III | Disodium EDTA | 0.01 | 0.01 |
| Natrosol 250 HHX | Hydroxyethyl- | 0.50 | 0.50 |
| | cellulose | | |
| Dehyton AB30 (30% | Coco-betaine | 6.0 | / |
| aqueous solution) | | | |
| Coco betaine | Coco-betaine | / | 6.0 × 0.3 = |
| active matter | | | 1.8 |
| (without water) | | | |
| Propylene glycol | Propylene glycol | 19.0 | 19.0 |
| Absolute ethanol | Ethanol | 30.4 | 30.4 |
| Nipanox BHT | Butyl hydroxy- | 0.04 | 0.04 |
| | toluene | | |
| Terbinafine HCl | Terbinafine HCl | 5.0 | 5.0 |

The initial pH is 4.75.

### Physical Stability:

| | Initial time | | 1 month | 2 months | 3 months |
|---|---|---|---|---|---|
| Macroscopic | Colourless | 4° C. | Idem | Idem | Idem |
| appearance | clear fluid | | Initial | initial | initial |
| | solution | | time | time | time |
| | | RT | Idem | Idem | Idem |
| | | | initial | initial | initial |
| | | | time | time | time |
| | | 40° C. | Idem | Idem | Idem |
| | | | initial | initial | initial |
| | | | time | time | time |
| Microscopic | Absence of | 4° C. | Idem | Idem | Idem |
| appearance | crystals | | initial | initial | initial |
| | | | time | time | time |
| | | RT | Idem | Idem | Idem |
| | | | initial | initial | initial |
| | | | time | time | time |

### Chemical Stability:

| | T0 | T 1 month | T 2 months | T 3 months |
|---|---|---|---|---|
| RT | 101.6% | 101.3% | 102.2% | 101.5% |
| 40° C. | — | 101% | 100.7% | 101.1% |

The composition from Example 2 is therefore physically and chemically stable over 3 months at 4° C., at room temperature and at 40° C.

### EXAMPLE 3

### Solution Containing 10% of Terbinafine HCl

| | | | Content |
|---|---|---|---|
| | | | (% taking into |
| | | | account the water |
| | | Content | contained in each |
| Ingredients | INCI name | (% w/w) | ingredient) |
| Purified water | Aqua | 29.45 | / |
| Total water | Aqua | / | 29.45 + |
| (purified water + | | | (6.0 × 0.7) = |
| water contained in | | | 33.65 |
| the ingredients) | | | |
| Titriplex III | Disodium EDTA | 0.01 | 0.01 |
| Klucel HF | Hydroxypropyl- | 0.5 | 0.5 |
| | cellulose | | |
| Dehyton AB30 (30% | Coco-betaine | 6.0 | / |
| aqueous solution) | | | |
| Coco betaine | Coco-betaine | / | 6.0 × 0.3 = |
| active matter | | | 1.8 |
| (without water) | | | |
| Propylene glycol | Propylene glycol | 20.25 | 20.25 |
| Absolute ethanol | Ethanol | 33.75 | 33.75 |
| Nipanox BHT | Butyl hydroxy- | 0.04 | 0.04 |
| | toluene | | |
| Terbinafine HCl | Terbinafine HCl | 10.0 | 10.0 |

The initial pH is 4.50.

### Physical Stability:

| | Initial time | | 1 month | 2 months | 3 months |
|---|---|---|---|---|---|
| Macroscopic | Colourless | 4° C. | Idem | Idem | Idem |
| appearance | clear fluid | | initial | initial | initial |
| | solution | | time | time | time |
| | | RT | Idem | Idem | Idem |
| | | | initial | initial | initial |
| | | | time | time | time |
| | | 40° C. | Idem | Idem | Idem |
| | | | initial | initial | initial |
| | | | time | time | time |
| Microscopic | Absence of | 4° C. | Idem | Idem | Idem |
| appearance | crystals | | initial | initial | initial |
| | | | time | time | time |
| | | RT | Idem | Idem | Idem |
| | | | initial | initial | initial |
| | | | time | time | time |

### Chemical Stability:

| | T0 | T 1 month | T 2 months | T 3 months |
|---|---|---|---|---|
| RT | 104% | 103.8% | 102.1% | 103.5% |
| 40° C. | — | 103.6% | 103% | 103.4% |

The composition from Example 3 is therefore physically and chemically stable over 3 months at 4° C., at room temperature and at 40° C.

### EXAMPLE 4

### Solution Containing 10% of Terbinafine

| | | | Content |
|---|---|---|---|
| | | | (% taking into |
| | | | account the water |
| | | Content | contained in each |
| Ingredients | INCI name | (% w/w) | ingredient) |
| Purified water | Aqua | 29.45 | / |
| Total water | Aqua | / | 29.45 + |
| (purified water + | | | (6.0 × 0.7) = |
| water contained in | | | 33.65 |
| the ingredients) | | | |
| Titriplex III | Disodium EDTA | 0.01 | 0.01 |
| Natrosol 250 HHX | Hydroxyethyl- | 0.5 | 0.5 |
| | cellulose | | |
| Dehyton AB30 (30% | Coco-betaine | 6.0 | / |
| aqueous solution) | | | |
| Coco betaine | Coco-betaine | / | 6.0 × 0.3 = |
| active matter | | | 1.8 |
| (without water) | | | |
| Propylene glycol | Propylene glycol | 20.25 | 20.25 |
| Absolute ethanol | Ethanol | 33.75 | 33.75 |
| Nipanox BHT | Butyl hydroxy- | 0.04 | 0.04 |
| | toluene | | |
| Terbinafine HCl | Terbinafine HCl | 10.0 | 10.0 |

The initial pH is 4.53.

### Physical Stability:

| | Initial time | | 1 month | 2 months | 3 months |
|---|---|---|---|---|---|
| Macroscopic | Colourless | 4° C. | Idem | Idem | Idem |
| appearance | clear fluid | | initial | initial | initial |
| | solution | | time | time | time |
| | | RT | Idem | Idem | Idem |
| | | | initial | initial | initial |
| | | | time | time | time |
| | | 40° C. | Idem | Idem | Idem |
| | | | initial | initial | initial |
| | | | time | time | time |
| Microscopic | Absence of | 4° C. | Idem | Idem | Idem |
| appearance | crystals | | initial | initial | initial |
| | | | time | time | time |
| | | RT | Idem | Idem | Idem |
| | | | initial | initial | initial |
| | | | time | time | time |

The composition from Example 4 is therefore physically stable for 3 months at 4° C., at room temperature and at 40° C.

### EXAMPLE 5

### Solution Containing 10% of Terbinafine

| | | | Content |
|---|---|---|---|
| | | | (% taking into |
| | | | account the water |
| | | Content | contained in each |
| Ingredients | INCI name | (% w/w) | ingredient) |
| Purified water | Aqua | 27.67 | / |
| Total water | Aqua | / | 27.67 + |
| (purified water + | | | (6.0 × 0.7) + |
| water contained in | | | 35.53 × 0.05) = |
| the ingredients) | | | 33.65 |
| Titriplex III | Disodium EDTA | 0.01 | 0.01 |
| Natrosol 250 HHX | Hydroxyethyl- | 0.50 | 0.50 |
| | cellulose | | |
| Dehyton AB30 (30% | Coco-betaine | 6.0 | / |
| aqueous solution) | | | |
| Coco betaine | Coco-betaine | / | 6.0 × 0.3 = |
| active matter | | | 1.8 |
| (without water) | | | |
| Propylene glycol | Propylene glycol | 20.25 | 20.25 |
| Ethanol 95-96% | Ethanol | 35.53 | / |
| Absolute ethanol | Ethanol | / | 35.53 − |
| | | | (35.53 × 0.05) = |
| | | | 33.75 |
| Nipanox BHT | Butyl hydroxy- | 0.04 | 0.04 |
| | toluene | | |
| Terbinafine HCl | Terbinafine HCl | 10.0 | 10.0 |

The initial pH is 4.36.

### Physical Stability:

| | Initial time | | 1 month | 2 months | 3 months |
|---|---|---|---|---|---|
| Macroscopic | Colourless | 4° C. | Idem | Idem | Idem |
| appearance | clear fluid | | initial | initial | initial |
| | solution | | time | time | time |
| | | RT | Idem | Idem | Idem |
| | | | initial | initial | initial |
| | | | time | time | time |
| | | 40° C. | Idem | Idem | Idem |
| | | | initial | initial | initial |
| | | | time | time | time |
| Microscopic | Absence of | 4° C. | Idem | Idem | Idem |
| appearance | crystals | | initial | initial | initial |
| | | | time | time | time |
| | | RT | Idem | Idem | Idem |
| | | | initial | initial | initial |
| | | | time | time | time |

### Chemical Stability:

| | T0 | T 1 month | T 2 months | T 3 months |
|---|---|---|---|---|
| RT | 100.9% | 101.4% | 100.3% | 98.6% |
| 40° C. | — | 101.2% | 99.1% | 99.2% |

The composition from Example 5 is therefore physically and chemically stable over 3 months at 4° C., at room temperature and at 40° C.

### EXAMPLE 6

### Solution Containing 10% of Terbinafine

| | | | Content |
|---|---|---|---|
| | | | (% taking into |
| | | | account the water |
| | | Content | contained in each |
| Ingredients | INCI name | (% w/w) | ingredient) |
| Purified water | Aqua | 27.87 | / |
| Total water | Aqua | / | 27.87 + |
| (purified water + | | | (6.0 × 0.7) + |
| water contained in | | | (35.53 × 0.05) = |
| the ingredients) | | | 33.85 |
| Titriplex III | Disodium EDTA | 0.01 | 0.01 |
| Natrosol 250 HHX | Hydroxyethyl- | 0.30 | 0.30 |
| | cellulose | | |
| Dehyton AB30 (30% | Coco-betaine | 6.0 | / |
| aqueous solution) | | | |
| Coco betaine | Coco-betaine | / | 6.0 × 0.3 = |
| active matter | | | 1.8 |
| (without water) | | | |
| Propylene glycol | Propylene glycol | 20.25 | 20.25 |
| Ethanol 95-96% | Ethanol | 35.53 | / |
| Absolute ethanol | Ethanol | / | 35.53 − |
| | | | (35.53 × 0.05) = |
| | | | 33.75 |
| Nipanox BHT | Butyl hydroxy- | 0.04 | 0.04 |
| | toluene | | |
| Terbinafine HCl | Terbinafine HCl | 10.0 | 10.0 |

The initial pH is 4.69.

### Physical Stability:

| | Initial time | | 1 month | 2 months | 3 months |
|---|---|---|---|---|---|
| Macroscopic | Colourless | 4° C. | Idem | Idem | Idem |
| appearance | clear fluid | | initial | initial | initial |
| | solution | | time | time | time |
| | | RT | Idem | Idem | Idem |
| | | | initial | initial | initial |
| | | | time | time | time |
| | | 40° C. | Idem | Idem | Idem |
| | | | initial | initial | initial |
| | | | time | time | time |
| Microscopic | Absence of | 4° C. | Idem | Idem | Idem |
| appearance | crystals | | initial | initial | initial |
| | | | time | time | time |
| | | RT | Idem | Idem | Idem |
| | | | initial | initial | initial |
| | | | time | time | time |

### Chemical Stability:

| | T0 | T 1 month | T 2 months | T 3 months |
|---|---|---|---|---|
| RT | 100.8% | 102.2% | 101.2% | 99.3% |
| 40° C. | — | 101.8% | 101% | 100.1% |

The composition from Example 6 is therefore physically and chemically stable over 3 months at 4° C., at room temperature and at 40° C.

### EXAMPLE 7

### Solution Containing 10% of Terbinafine

| | | | Content |
|---|---|---|---|
| | | | (% taking into |
| | | | account the water |
| | | Content | contained in each |
| Ingredients | INCI name | (% w/w) | ingredient) |
| Purified water | Aqua | 26.67 | / |
| Total water | Aqua | / | 26.67 + |
| (purified water + | | | (6.0 × 0.7) + |
| water contained in | | | (35.53 × 0.05) = |
| the ingredients) | | | 32.65 |
| Titriplex III | Disodium EDTA | 0.01 | 0.01 |
| Natrosol 250 HHX | Hydroxyethyl- | 0.50 | 0.50 |
| | cellulose | | |
| Dehyton AB30 (30% | Coco-betaine | 6.0 | / |
| aqueous solution) | | | |
| Coco betaine | Coco-betaine | / | 6.0 × 0.3 = |
| active matter | | | 1.8 |
| (without water) | | | |
| Propylene glycol | Propylene glycol | 20.25 | 20.25 |
| Ethanol 95-96% | Ethanol | 35.53 | / |
| Absolute ethanol | Ethanol | / | 35.53 − |
| | | | (35.53 × 0.05) = |
| | | | 33.75 |
| Nipanox BHT | Butyl hydroxy- | 0.04 | 0.04 |
| | toluene | | |
| Terbinafine HCl | Terbinafine HCl | 10.0 | 10.0 |
| Acetic acid | Acetic acid | 1.0 | 1.0 |

The initial pH is 3.98.

### Physical Stability:

| | Initial time | | 1 month | 2 months | 3 months |
|---|---|---|---|---|---|
| Macroscopic | Colourless | 4° C. | Idem | Idem | Idem |
| appearance | clear fluid | | initial | initial | initial |
| | solution | | time | time | time |
| | | RT | Idem | Idem | Idem |
| | | | initial | initial | initial |
| | | | time | time | time |
| | | 40° C. | Idem | Idem | Idem |
| | | | initial | initial | initial |
| | | | time | time | time |
| Microscopic | Absence of | 4° C. | Idem | Idem | Idem |
| appearance | crystals | | initial | initial | initial |
| | | | time | time | time |
| | | RT | Idem | Idem | Idem |
| | | | initial | initial | initial |
| | | | time | time | time |

### Chemical Stability:

| | T0 | T 15 days |
|---|---|---|
| RT | 100.1% | / |
| 40° C. | — | 101.2% |

The composition from Example 7 is therefore physically and chemically stable for 15 days at 4° C., at room temperature and at 40° C.

### EXAMPLE 8

### Solution Containing 10% of Terbinafine

| | | | Content |
|---|---|---|---|
| | | | (% taking into |
| | | | account the water |
| | | Content | contained in each |
| Ingredients | INCI name | (% w/w) | ingredient) |
| Purified water | Aqua | 26.67 | / |
| Total water | Aqua | / | 26.67 + |
| (purified water + | | | (6.0 × 0.7) + |
| water contained in | | | (35.53 × 0.05) = |
| the ingredients) | | | 32.65 |
| Titriplex III | Disodium EDTA | 0.01 | 0.01 |
| Natrosol 250 HHX | Hydroxyethyl- | 0.50 | 0.50 |
| | cellulose | | |
| Dehyton AB30 (30% | Coco-betaine | 6.0 | / |
| aqueous solution) | | | |
| Coco betaine | Coco-betaine | / | 6.0 × 0.3 = |
| active matter | | | 1.8 |
| (without water) | | | |
| Propylene glycol | Propylene glycol | 16.25 | 16.25 |
| Ethanol 95-96% | Ethanol | 35.53 | / |
| Absolute ethanol | Ethanol | / | 35.53 − |
| | | | (35.53 × 0.05) = |
| | | | 33.75 |
| Nipanox BHT | Butyl hydroxy- | 0.04 | 0.04 |
| | toluene | | |
| Terbinafine HCl | Terbinafine HCl | 10.0 | 10.0 |
| ST Cyclomethicone | Cyclomethicone | 5.0 | 5.0 |
| 5 | | | |

### Physical Stability:

| | Initial time | | 1 month | 2 months | 3 months |
|---|---|---|---|---|---|
| Macroscopic | Colourless | 4° C. | Idem | Idem | Idem |
| appearance | clear fluid | | initial | initial | initial |
| | solution | | time | time | time |
| | | RT | Idem | Idem | Idem |
| | | | initial | initial | initial |
| | | | time | time | time |
| | | 40° C. | Idem | Idem | Idem |
| | | | initial | initial | initial |
| | | | time | time | time |
| Microscopic | Absence of | 4° C. | Idem | Idem | Idem |
| appearance | crystals | | initial | initial | initial |
| | | | time | time | time |
| | | RT | Idem | Idem | Idem |
| | | | initial | initial | initial |
| | | | time | time | time |

The composition from Example 8 is therefore physically stable over 3 months at 4° C., at room temperature and at 40° C.

### EXAMPLE 9

### Solution Containing 10% of Terbinafine

| | | | Content |
|---|---|---|---|
| | | | (% taking into |
| | | | account the water |
| | | Content | contained in each |
| Ingredients | INCI name | (% w/w) | ingredient) |
| Purified water | Aqua | 31.59 | / |
| Total water | Aqua | / | 31.59 + |
| (purified water + | | | (6.0 × 0.7) + |
| water contained in | | | (33.16 × 0.05) = |
| the ingredients) | | | 37.45 |
| Titriplex III | Disodium EDTA | 0.01 | 0.01 |
| Natrosol 250 HHX | Hydroxyethyl- | 0.3 | 0.3 |
| | cellulose | | |
| Dehyton AB30 (30% | Coco-betaine | 6.0 | / |
| aqueous solution) | | | |
| Coco betaine | Coco-betaine | / | 6.0 × 0.3 = |
| active matter | | | 1.8 |
| (without water) | | | |
| Propylene glycol | Propylene glycol | 18.9 | 18.9 |
| Ethanol 95-96% | Ethanol | 33.16 | / |
| Absolute ethanol | Absolute ethanol | / | 33.16 − |
| | | | (33.16 × 0.05) = |
| | | | 31.50 |
| Nipanox BHT | Butyl hydroxy- | 0.04 | 0.04 |
| | toluene | | |
| Terbinafine HCl | Terbinafine HCl | 10.0 | 10.0 |

The initial pH is 4.52.

### Physical Stability:

| | Initial time | | 1 month | 2 months | 3 months |
|---|---|---|---|---|---|
| Macroscopic | Colourless | 4° C. | Idem | Idem | Idem |
| appearance | clear fluid | | initial | initial | initial |
| | solution | | time | time | time |
| | | RT | Idem | Idem | Idem |
| | | | initial | initial | initial |
| | | | time | time | time |
| | | 40° C. | Idem | Idem | Idem |
| | | | initial | initial | initial |
| | | | time | time | time |
| Microscopic | Absence of | 4° C. | Idem | Idem | Idem |
| appearance | crystals | | initial | initial | initial |
| | | | time | time | time |
| | | RT | Idem | Idem | Idem |
| | | | initial | initial | initial |
| | | | time | time | time |

### Chemical Stability:

| | T0 | T 1 month | T 2 months | T 3 months |
|---|---|---|---|---|
| RT | 100.1% | 99.9% | 99% | 100.8% |
| 40° C. | — | 99.2% | 99.5% | 101.8% |

The composition from Example 9 is therefore physically and chemically stable over 3 months at 4° C., at room temperature and at 40° C.

It appears that all of the formulations tested (Examples 2 to 9) are stable both from a physical and chemical point of view, despite the variation in the various parameters:
the concentration of active principle, in this case terbinafine HCl;
the optional addition of antiseptic agent(s) such as acetic acid;
the nature or concentration of the texturing agent; and
the composition of the ternary solvent system, in particular in the proportion of the three respective constituents.

### EXAMPLE 10A

### Solution Containing 10% of Terbinafine

| | | | Content |
|---|---|---|---|
| | | | (% taking into |
| | | | account the water |
| | | Content | contained in each |
| Ingredients | INCI name | (% w/w) | ingredient) |
| Purified water | Aqua | 33.495 | / |
| Total water | Aqua | / | 33.495 + |
| (purified water + | | | (0.375 × 0.7) + |
| water contained in | | | (35.53 × 0.05) = |
| the ingredients) | | | 35.54 |
| Titriplex III | Disodium EDTA | 0.01 | 0.01 |
| Natrosol 250 HHX | Hydroxyethyl- | 0.30 | 0.30 |
| | cellulose | | |
| Dehyton AB30 (30% | Coco-betaine | 0.375 | / |
| aqueous solution) | | | |
| Coco betaine | Coco-betaine | / | 0.375 × 0.3 = |
| active matter | | | 0.11 |
| (without water) | | | |
| Propylene glycol | Propylene glycol | 20.25 | 20.25 |
| Ethanol 95-96% | Ethanol | 35.53 | / |
| Absolute ethanol | Ethanol | / | 35.53 − |
| | | | (35.53 × 0.05) = |
| | | | 33.75 |
| Nipanox BHT | Butyl hydroxy- | 0.04 | 0.04 |
| | toluene | | |
| Terbinafine HCl | Terbinafine HCl | 10.0 | 10.0 |

### EXAMPLE 10B

### Solution Containing 10% of Terbinafine

| | | | Content |
|---|---|---|---|
| | | | (% taking into |
| | | | account the water |
| | | Content | contained in each |
| Ingredients | INCI name | (% w/w) | ingredient) |
| Purified water | Aqua | 33.537 | / |
| Total water | Aqua | / | 33.537 + |
| (purified water + | | | (0.333 × 0.7) + |
| water contained in | | | (35.53 × 0.05) = |
| the ingredients) | | | 35.42 |
| Titriplex III | Disodium EDTA | 0.01 | 0.01 |
| Natrosol 250 HHX | Hydroxyethyl- | 0.30 | 0.30 |
| | cellulose | | |
| Dehyton AB30 (30% | Coco-betaine | 0.333 | / |
| aqueous solution) | | | |
| Coco betaine | Coco-betaine | / | 0.333 × 0.3 = |
| active matter | | | 0.1 |
| (without water) | | | |
| Propylene glycol | Propylene glycol | 20.25 | 20.25 |
| Ethanol 95-96% | Ethanol | 35.53 | / |
| Absolute ethanol | Ethanol | / | 35.53 − |
| | | | (35.53 × 0.05) = |
| | | | 33.75 |
| Nipanox BHT | Butyl hydroxy- | 0.04 | 0.04 |
| | toluene | | |
| Terbinafine HCl | Terbinafine HCl | 10.0 | 10.0 |

### EXAMPLE 11

### Solution Containing 10% of Terbinafine

| | | | Content |
|---|---|---|---|
| | | | (% taking into |
| | | | account the water |
| | | Content | contained in each |
| Ingredients | INCI name | (% w/w) | ingredient) |
| Purified water | Aqua | 32.37 | / |
| Total water | Aqua | / | 32.37 + |
| (purified water + | | | (1.50 × 0.7) + |
| water contained in | | | (35.53 × 0.05) = |
| the ingredients) | | | 35.20 |
| Titriplex III | Disodium EDTA | 0.01 | 0.01 |
| Natrosol 250 HHX | Hydroxyethyl- | 0.30 | 0.30 |
| | cellulose | | |
| Dehyton AB30 (30% | Coco-betaine | 1.50 | / |
| aqueous solution) | | | |
| Coco betaine | Coco-betaine | / | 1.50 × 0.3 = |
| active matter | | | 0.45 |
| (without water) | | | |
| Propylene glycol | Propylene glycol | 20.25 | 20.25 |
| Ethanol 95-96% | Ethanol | 35.53 | / |
| Absolute ethanol | Ethanol | / | 35.53 − |
| | | | (35.53 × 0.05) = |
| | | | 33.75 |
| Nipanox BHT | Butyl hydroxy- | 0.04 | 0.04 |
| | toluene | | |
| Terbinafine HCl | Terbinafine HCl | 10.0 | 10.0 |

### EXAMPLE 12

### Study of Keratin Binding

The objective is to demonstrate the effect of the addition, in a formulation containing 10% of terbinafine hydrochloride, of an amphoteric agent such as coco betaine on the affinity of the terbinafine hydrochloride for the keratin of the nail.

Two formulations are compared:
Formulation 1: control formulation that does not contain amphoteric agent; and
Formulation 2: formulation containing 1.8% of coco betaine, i.e. 6% of a 30% aqueous solution of coco betaine corresponding to Example 9.

The concentration of terbinafine hydrochloride is identical in both formulations (10% w/w). Their exact composition is presented below:

| | Formula 1 (control) | Formula 2 |
|---|---|---|
| Ingredients | (% w/w) | (% w/w) |
| Purified water | 37.59 | 31.59 |
| Titriplex III | 0.01 | 0.01 |
| Natrosol 250 HHX | 0.30 | 0.30 |
| Dehyton AB30 | / | 6.0 |
| (30% aqueous solution) | | |
| Propylene glycol | 18.9 | 18.9 |
| 95% of ethanol | 33.16 | 33.16 |
| Nipanox BHT | 0.04 | 0.04 |
| Terbinafine HCl | 10.0 | 10.0 |

In the preparations tested, due to the presence of 10% terbinafine hydrochloride, the pH is acid. The amphoteric agent present in the solution is then predominantly in cationic form. Since the keratin of the nail is negatively charged, it is a question of verifying that the amphoteric agent in cationic form binds preferentially to the keratin, saturating the binding sites and then enabling the terbinafine hydrochloride to be free and available in order to reach its site of action: the nail bed.

The adsorption kinetics of terbinafine hydrochloride on powdered keratin were monitored over time in order to demonstrate the competition of the amphoteric agent in cationic form and of the terbinafine hydrochloride with respect to the keratin. In order to do this, a procedure was developed from studies by Tatsumi et al. (Therapeutic efficacy of topically applied KP-103 against experimental tinea unguium in guinea pigs in comparison with Amorolfine and terbinafine, Antimicrobial agents and chemotherapy, (2002), 46, 3797-3801): the formulation is brought into contact with the keratin powder. The mixture is then centrifuged, then the concentration of free terbinafine hydrochloride remaining in the formulation is measured at various times.

### 1/Sample Preparation

In order not to be under conditions of total saturation of the keratin by the terbinafine hydrochloride, the preparations tested are diluted to 1/100^{th} in the corresponding placebo. The pH is then adjusted to 3.6 in order to remain under the native pH conditions of the preparation.

The keratin is then added (10% w/w) and the whole mixture is stirred magnetically at 25° C. At the time of analysis, 1 ml of preparation is withdrawn and centrifuged for 10 min at 13 000 rpm. The supernatant is then assayed by HPLC.

### 2/HPLC Assay Method

The terbinafine hydrochloride is analysed at 283 nm on a Sunfire C18 column (150×4.6 mm, 3.5 μm particles) in isocratic mode (1.5 ml/min) with an acetonitrile/methanol/buffer solution mixture (700/200/100 v/v/v). The buffer is prepared with 1000 ml of water and 1 ml of triethylamine and is adjusted to pH 7.5. The controls and samples are prepared at a concentration of 0.05 mg/ml of terbinafine hydrochloride by dilution in an acetonitrile/methanol/Milli-Q water phase (700/200/100 v/v/v).

### 3/Results

The results of the adsorption kinetics are listed in the table below which shows the variations of the terbinafine hydrochloride content relative to the initial content (% Label Claim) as a function of the time of the test formula (1.8% coco betaine, i.e. 6% of a 30% solution of coco betaine in water) and of the control formula (0% coco betaine):

| | Formula 1 (control) | | Formula 2 (Example 9) | |
|---|---|---|---|---|
| Time (min) | % LC | CV % | % LC | CV % |
| 0 | 84.4 | 0.5 | 98.6 | 0.3 |
| 30 | 74.7 | 3.8 | 94.9 | 1.1 |
| 60 | 71.9 | 3.2 | 93.3 | 1.8 |
| 120 | 71.0 | 3.0 | 90.6 | 0.9 |
| 240 | 68.9 | 2.9 | 89.4 | 0.7 |
| 360 | 67.7 | 1.7 | 89.2 | 1.3 |

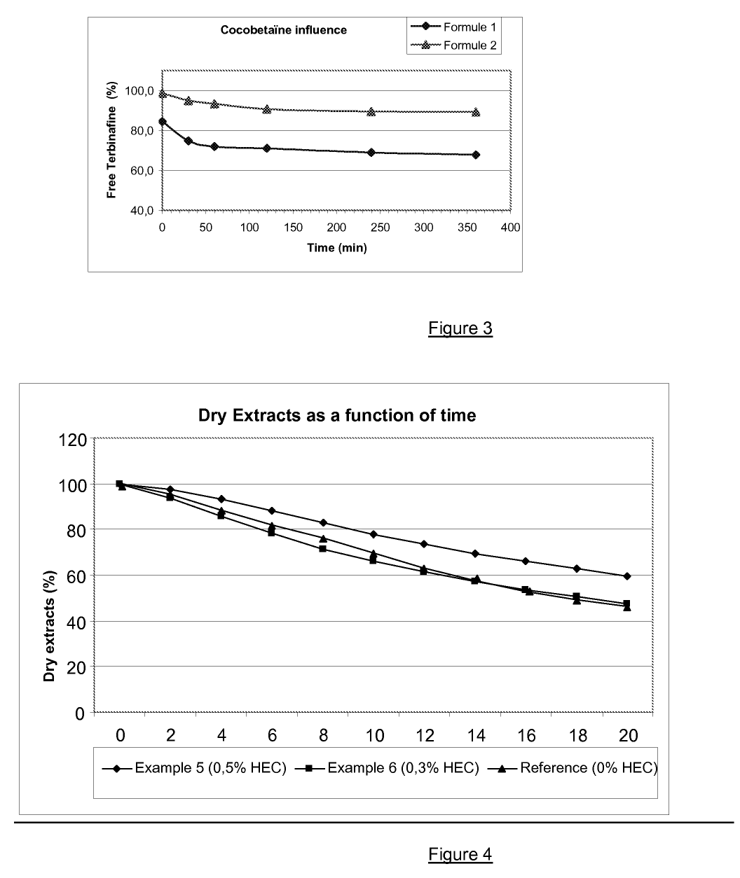

The corresponding graph is presented in FIG. 3.

In the absence of coco betaine (Formula 1), an immediate binding of the terbinafine hydrochloride to the keratin is observed. The phenomenon continues until it rapidly reaches a plateau corresponding to the saturation of the keratin and around 30% of terbinafine hydrochloride bound to the keratin.

In the presence of 1.8% of coco betaine, i.e. 6% of a 30% solution of coco betaine in water (Formula 2), the adsorption of the terbinafine hydrochloride on the keratin is greatly minimized. Indeed, at the initial time, the adsorption is negligible for formulation 2 (98.6% of free terbinafine). At the plateau, the presence of coco betaine makes it possible to increase the bioavailability of the terbinafine hydrochloride by more than 20%.

The addition of coco betaine to formulation 2 therefore enables competition with the terbinafine hydrochloride with respect to the adsorption on keratin. The concentration of free terbinafine in formulation 2 is therefore greater, rendering the terbinafine more available.

In conclusion, the addition of an amphoteric agent to the formulation makes it possible, under the acid pH conditions of the preparation, that is to say at pH values below the pKa of the terbinafine hydrochloride (7.10) and preferably at pH values between 3 and 5, to render the terbinafine hydrochloride more available. Indeed, the binding of the latter to the keratin of the nail is reduced owing to a phenomenon of competition with the amphoteric agent in cationic form. In this way, the active principle is then more available for reaching its site of action, which is the nail bed.

### EXAMPLE 13

### Influence of the Texturing Agent Content

This study consists in monitoring the evaporation kinetics in order to study the effect of the content of texturing agent.

The evaporation kinetics of various compositions were tested at 32° C., which corresponds to the temperature of the skin and of the nail.

### 1/Operating Conditions

### Equipment:

SARTORIUS Type MA100 moisture analyser Infrared radiation enables the evaporation of the volatile components contained in the product.

### Method:

| Parameters | Values |
|---|---|
| Heating programme | Standard drying |
| Start of the analysis | With stability |
| End of the analysis | Time = 20 min |
| Weight resolution value for analysis | 1 mg |
| Results | Dry weight (% R) |
| Print interval of the values | 2 min |

The formulations tested correspond to those from Examples 5 and 6. The reference used corresponds to a formulation with no texturing agent and with no coco betaine as described below:

| Ingredients | Reference (% w/w) |
|---|---|
| Purified water | 35.95 |
| Titriplex III | 0.01 |
| Propylene glycol | 20.25 |
| Absolute ethanol | 33.75 |
| Nipanox BHT | 0.04 |
| Terbinafine HCl | 10.0 |

### 2/Results:

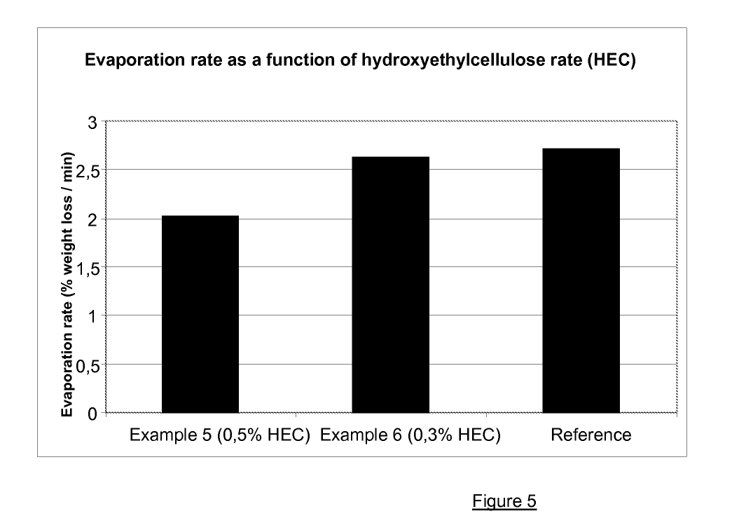

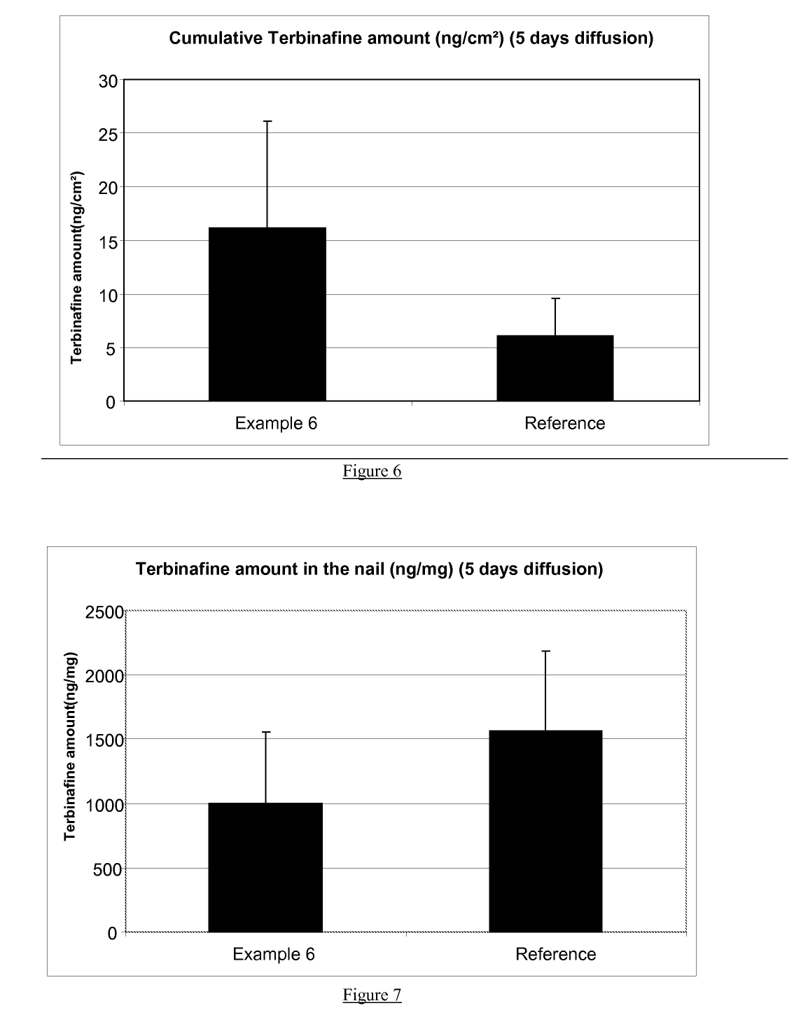

The corresponding results are presented in FIGS. 4 and 5.

The evaporation kinetics and also the evaporation rate of the formula from Example 6 (0.3% hydroxyethyl cellulose) are equivalent to those of the reference formula. On the other hand, the addition of 0.5% of hydroxyethyl cellulose to the composition (Example 5) reduces the evaporation kinetics.

The content of 0.3% hydroxyethyl cellulose (Example 6) is optimized and makes it possible to obtain evaporation kinetics identical to those of a composition with no texturing agent.

### EXAMPLE 14

### Study of the Diffusion of Terbinafine HCl Through the Nail in the Presence and in the Absence of Amphoteric Agent

The objective of this study is to compare in vitro the penetration through nails of two formulations containing 10% of terbinafine hydrochloride with and without coco betaine.

The treatment time is 5 days with a new application of each formula each day. The concentration of formulation deposited daily on the surface of the nail is 10 μl/cm².

The formulations tested correspond to those of:
Example 6, formulation containing coco betaine; and
the reference described in Example 11, formulation not containing coco betaine.

### 1/Operating Conditions:

Nails: originating from the hands of human cadavers (6 nails for each formulation tested).

Diffusion setup: composed of Franz diffusion cells (diameter: 7 mm) and a bath thermostatically controlled at 32° C.

Receiving liquid: 1/10 phosphate buffer (pH 7.4±0.1) containing 0.1% of Volpo (Oleth-20) (PBS diluted to 1:10, to which 0.1% of Volpo is added).

The diffusion cells used in this study have a diameter of 7 mm. The nails are placed in the diffusion cell with the ventral side towards the receiving compartment. The leaktightness is ensured by a hard silicone seal.

During the experiment, the diffusion cells are placed in a thermostatically-controlled bath in order to obtain a temperature of 32±1° C. at the surface of the nail. This temperature is checked before each withdrawal.

The first day (D0), the diffusion cells on which the nails are mounted are placed in a thermostatically-controlled bath. The receiving compartment is filled with the receiving liquid and the surface of the nail is left in the open air. The next day (D1), all of the receiving liquid is withdrawn from all of the diffusion cells and is replaced by a fresh solution of receiving liquid. Next, 10 μl/cm² of each formulation are applied to the surface of the nails. The application is repeated every 24 h for 5 days following the same procedure as on day 1 (10 μl/cm² of each formulation are applied to the surface of the nails). Before each application, all of the receiving liquid is withdrawn from all of the diffusion cells and is replaced with a fresh solution of receiving liquid. Before filling with the fresh solution of receiving liquid, the surface of the nails is cleaned with a solution of 1% sodium lauryl sulphate (SLS) and demineralized water.

The last day (D6), after the last withdrawal, the nails are removed from the diffusion cell and the surface is cleaned with a solution of 1% SLS then rinsed with demineralized water. The nails are then cut up and put into acetone for 24 hours with stirring. The acetone is withdrawn and evaporated to dryness under a fume hood.

The samples, receiving liquid and nail extract are then analysed by MS/MS.

### 2/Results

Accumulated amount of terbinafine HCl in the receiving liquid (ng·cm⁻²)

| | | | | | | | | Stan- |
|---|---|---|---|---|---|---|---|---|
| | | | | | | | | dard |
| Time | | | | | | | Av- | devi- |
| (days) | | | | | | | erage | ation |
| | Example 6 formulation | | | | | | | |
| Samples | A1 | B1 | C1 | D1 | E1 | F1 | | |
| 5 days | 13.88 | 33.46 | 20.82 | 4.39 | 12.11 | 12.07 | 16.12 | 9.98 |
| | Reference formulation | | | | | | | |
| Samples | A2 | B2 | C2 | D2 | E2 | F2 | | |
| 5 days | 8.78 | 5.30 | 5.19 | 0.00 | 8.78 | 8.60 | 6.11 | 3.44 |

After 5 days of diffusion, the amount of terbinafine hydrochloride that has diffused through the nail from the formulation containing the amphoteric agent is 2.7 times greater than that which does not contain any thereof.

Terbinafine in the nail (ng·mg⁻²)

| | | | | | | | Standard |
|---|---|---|---|---|---|---|---|
| | | | | | | Average | deviation |
| Example 6 formulation | | | | | | | |
| A1 | B1 | C1 | D1 | E1 | F1 | | |
| 745.22 | 1112.48 | 614.58 | 488.23 | 2036.01 | 984.83 | 996.89 | 558.84 |
| Reference formulation | | | | | | | |
| A2 | B2 | C2 | D2 | E2 | F2 | | |
| 2448.72 | 1910.61 | 1262.61 | 612.43 | 1536.14 | 1615.51 | 1564.34 | 616.87 |

The amounts of terbinafine hydrochloride in the nail are lower for the formula that contains the amphoteric agent. These results show that the terbinafine hydrochloride in the presence of the amphoteric agent binds less in the nail and therefore is more available for diffusing through the nail matrix.

A contrario, the terbinafine hydrochloride in the absence of the amphoteric agent binds in a larger amount in the nail thus creating a reservoir effect that limits the diffusion of the terbinafine hydrochloride to the receiving liquid.

1. A method of preparing a composition for application to a nail, the method comprising preparing the composition with an effective amount of a cationic surfactant or of an amphoteric surfactant wherein the composition is in the form of a solution comprising an antifungal agent in the form of an acid salt.

2. The method as defined in claim 1, wherein the composition does not contain any film-forming agent.

3. A pharmaceutical composition comprising:
an antifungal agent that is in the form of an acid salt;
a solvent system; and
a cationic surfactant, wherein
said composition is in the form of a solution.

4. The pharmaceutical composition as defined in claim 3, wherein the composition is in the form of a solution that does not contain any film-forming agent.

5. The composition as defined by claim 3, wherein the pH of the composition is from 3 to 6.

6. The composition as defined by claim 3, wherein the amphoteric surfactant is a derivative of betaine.

7. The composition as defined by claim 3, wherein the surfactant represents from 0.1% to 20% by weight of the composition.

8. The composition as defined by claim 3, wherein the composition further comprises a texturing agent.

9. The composition as defined by claim 3, wherein the antifungal agent is an allylamine or a morpholine.

10. The composition as defined by claim 3, wherein the antifungal agent represents at least 5% of the composition by weight.

11. The composition as defined by claim 3, wherein the solvent system comprises:
water;
at least one C2-C8 alkanol with a straight or branched chain; and
at least one glycol.

12. The composition as defined by claim 3, wherein the composition comprises at least one compound selected from the group consisting of a chelating agent, an antioxidant, an antiseptic, and an emollient.

13. The method as defined by claim 3, wherein the composition is a medicinal product formulated to be applied to the nail for the treatment of onychomycoses.

14. The method of claim 1, wherein the antifungal agent is a hydrochloride.

15. The composition of claim 3, wherein the antifungal agent is a hydrochloride.

16. The composition of claim 3, wherein the cationic surfactant is a positively charged amphoteric surfactant.

17. The composition of claim 5, wherein the pH of the composition is from 3 to 5.

18. The composition of claim 6, wherein the derivative of betaine is coco betaine.

19. The composition of claim 7, wherein the surfactant is from 0.1% to 15% by weight of the composition.

20. The composition of claim 7, wherein the surfactant is from 0.1% to 10% by weight of the composition.

21. The composition of claim 8, wherein the texturing agent is an alkyl cellulose.

22. The composition as defined by claim 9, wherein the antifungal agent is terbinafine.

23. The composition of claim 10, wherein the antifungal agent represents at least 8% of the composition by weight.

24. The composition of claim 10, wherein the antifungal agent represents at least 10% of the composition by weight.

25. The composition of claim 11, wherein the at least one C2-C8 alkanol is ethanol.

26. The composition of claim 11, wherein the at least one glycol is propylene glycol.
